(19) United States
(12) Patent Application Publication
(10) Pub. No.: US 2025/0304618 A1
(43) Pub. Date: Oct. 2, 2025

(54) METHODS FOR UNIFIED CONCENTRATION AND BUFFER EXCHANGE

(71) Applicant: AstraZeneca AB, Södertälje (SE)

(72) Inventors: Jon COFFMAN, Gaithersburg, MD (US); Ujwal PATIL, Gaithersburg, MD (US); Irina RAMOS, Gaithersburg, MD (US)

(21) Appl. No.: 18/872,994

(22) PCT Filed: Jun. 9, 2023
(86) PCT No.: PCT/IB2023/055976

§ 371 Date: Dec. 9, 2024

Related U.S. Application Data
(60) Provisional application No. 63/366,147, filed on Jun. 10, 2022.

(51) Int. Cl. C07K 1/34; C07K 16/00

(52) U.S. Cl. CPC C07K 1/34; C07K 16/00; C07K 2319/00

(57) ABSTRACT

Provided herein are methods of purifying proteins of interest using asymmetric dialysis.

### CROSS-REFERENCE TO RELATED APPLICATIONS

This application claims priority benefit of U.S. Provisional Patent Application No. 63/366,147, filed Jun. 10, 2022, which is incorporated by reference herein in its entirety for all purposes.

### BACKGROUND OF THE DISCLOSURE

As biologics move to the forefront of drug development, the need for improved manufacturing processes has grown. With increasing projected demands for recombinant protein therapeutics, more cost effective and flexible manufacturing processes are needed. Indeed, various economic analyses estimate that process development and clinical manufacturing costs can constitute 40-60 percent of a drug's development cost. Along with commercial manufacturing, driven largely by downstream processing of consumable material costs, this can reach up to 25 percent of the sales revenue for a biologic. Accordingly, there is a need for more efficient downstream processing.

### SUMMARY OF THE DISCLOSURE

The present disclosure is direct to a method for purifying a protein of interest using counter-current concentration dialysis, comprising: (a) passing a first flow solution comprising the protein of interest and impurities into a first hollow fiber dialysis cassette at a first flow rate, wherein the dialysis cassette comprises a dialysate in-flow, at a dialysate in-flow rate, and a dialysate out-flow, at a dialysate out-flow rate; and wherein the first flow solution is counter-current to the dialysate in-flow and out-flow; (b) passing the impurities through a semi-permeable membrane of the dialysis cassette, wherein the dialysate in-flow rate is higher than the first flow rate, wherein a second flow solution comprising the protein of interest and a reduced level of impurities exits the dialysis cassette at a second flow rate, and wherein the dialysate out-flow rate is the sum of the dialysate in-flow rate and the difference between the first flow rate and the second flow rate; (c) optionally passing the second flow solution from the first dialysis cassette directly into a second dialysis cassette; and (d) optionally repeating steps (a) and (b) with the second flow solution and the second dialysis cassette, thereby forming a third flow solution with a reduced level of impurities compared to the first and second flow solutions.

In one aspect, the method further comprises passing the third flow solution from the second dialysis cassette directly into a third dialysis cassette, and repeating steps (a) and (b), thereby forming a fourth flow solution with a reduced level of impurities compared to the first, second, and third flow solutions. In another aspect, the method further comprises passing the fourth flow solution from the third dialysis cassette directly into a fourth dialysis cassette, and repeating steps (a) and (b), thereby forming a fifth flow solution with a reduced level of impurities compared to the first, second, third, and fourth flow solutions.

In one aspect, the dialysate in-flow rate is about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 7, about 1.8, about 1.9, about 2.0, about 2.1, about 2.2, about 2.25, about 2.3, about 2.4, about 2.5, about 2.6, about 2.7, about 2.8, about 2.9, about 3, about 4, about 5, about 6, about 7, about 8, about 9, or about 10 times higher than the first flow rate. In another aspect, the dialysate in-flow rate is about 2.25 times higher than the first flow rate. In another aspect, the second flow rate is about 0.1, about 0.15, about 0.2, about 0.25, about 0.3, about 0.35, about 0.4, about 0.45, about 0.5, about 0.55, about 0.6, about 0.65, about 0.7, or about 0.75 times the first flow rate. In another aspect, the second flow rate is between about 0.25 to about 0.5 times the first flow rate. In another aspect, the first flow rate is between about 0.01 mL/minute to about 25 mL/minute. In another aspect, the first flow rate is about 0.5 mL/minute, about 1 mL/minute, about 2 mL/minute, about 3 mL/minute, about 4 mL/minute, about 5 mL/minute, about 6 mL/minute, about 7 mL/minute, about 8 mL/minute, about 9 mL/minute, about 10 mL/minute, about 11 mL/minute, about 12 mL/minute, about 13 mL/minute, about 14 mL/minute, about 15 mL/minute, about 16 mL/minute, about 17 mL/minute, about 18 mL/minute, about 19 mL/minute, about 20 mL/minute, about 21 mL/minute, about 22 mL/minute, about 23 mL/minute, about 24 mL/minute, or about 25 mL/minute.

In one aspect, the impurities comprise low molecular weight species. In another aspect, the low molecular weight species are ionic impurities, such as salts of inorganic acids/bases, and other species (amino acids), culture additives, metal salts, carbohydrates (<1000 kDa), and chelating agents, such as EDTA.

In one aspect, the protein of interest is diafiltrated.

In one aspect, the protein of interest is obtained from a bioreactor.

In one aspect, about 0.1 kg/day, about 0.5 kg/day, about 1 kg/day, about 2 kg/day, about 3 kg/day, about 4 kg/day, about 5 kg/day, about 6 kg/day, about 7 kg/day, about 8 kg/day, about 9 kg/day or about 10 kg/day of protein of interest is purified.

In one aspect, the protein of interest comprises an antibody, an antigen binding fragment, a fusion protein, a naturally occurring protein, a chimeric protein, or any combination thereof. In another aspect, the protein comprises an antibody selected from IgM, IgA, IgE, IgD, and IgG. In another aspect, the protein comprises an antibody and the antibody is an IgG antibody selected from IgG1, IgG2, IgG3, and IgG4. In yet another aspect, the antibody is a therapeutic antibody.

### BRIEF DESCRIPTION OF THE DRAWINGS

FIG. 1 shows a schematic diagram of a single-pass asymmetric dialysis system. The feed containing monoclonal antibody (mAb) is pumped into the hollow-fiber module using pump (P1). The concentration factor along the cartridge is modulated using pumps, P2 and P4. The fresh dialysis buffer is delivered to the shell-side using pump P3.

FIG. 2 shows the relationship between a′ and buffer consumption per gram of mAb for asymmetric dialysis of 20 g/L mAb feed with target concentration factor of 10×.

FIG. 3 shows a schematic of a complete continuous downstream process incorporating asymmetric dialysis.

### DETAILED DESCRIPTION

The present disclosure provides a highly effective approach to remove contaminants during protein purification using asymmetric continuous counter-current concentration dialysis in series, without the need for chromatography. As such, the present disclosure provides methods for purifying a protein of interest that uses approximately 1/10^{th} the amount of water and solutions as chromatographic processes.

### I. Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation of modification, such as conjugation with a labeling component. Also included in the definition are, for example, polypeptides containing one or more analogs of an amino acid (including, for example, unnatural amino acids, etc.), as well as other modifications known in the art. The term “polypeptide” and “protein” as used herein specifically encompass antibodies and Fc domain-containing polypeptides (e.g., immunoadhesins).

As used herein, the term “protein of interest” is used in its broadest sense to include any protein (either natural or recombinant), present in a mixture, for which purification is desired. Such proteins of interest include, without limitation, enzymes, hormones, growth factors, cyotokines, immunoglobulins (e.g., antibodies), and/or any fusion proteins. In some aspects, the protein of interest refers to any protein that can be produced by the methods described herein. In some aspects, the protein of interest is an antibody. In some aspects, the protein of interest is a recombinant protein.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

As used herein the term “impurity” is used in its broadest sense to cover any undesired component, contaminant, or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants. In certain embodiments, the contaminant precipitate is separated from the cell culture using another means, such as centrifugation, sterile filtration, depth filtration and tangential flow filtration.

The term “HMW Species” refers to any one or more unwanted proteins present in a mixture. High molecular weight species can include dimers, trimers, tetramers, or other multimers. These species are often considered product related impurities, and can either be covalently or non-covalently linked, and can also, for example, consist of misfolded monomers in which hydrophobic amino acid residues are exposed to a polar solvent, and can cause aggregation.

The term “LMW Species” refers to any one or more unwanted species present in a mixture. Low molecular weight species are often considered product related impurities, and can include clipped species, or half molecules for compounds intended to be dimeric (such as monoclonal antibodies).

The term “Host Cell Proteins” or HCP refers to the undesirable proteins generated by a host cell unrelated to the production of the intended protein of interest. Undesirable host cell proteins can be secreted into the upstream cell culture supernatant. Undesirable host cell proteins can also be released during cell lysis. The cells used for upstream cell culture require proteins for growth, transcription, and protein synthesis, and these unrelated proteins are undesirable in a final drug product.

The term “fed-batch culture” or “fed-batch culture process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system.

The term “ultrafiltration” refers to, for example, a membrane-based separation process that separates molecules in solution based on size, which can accomplish separation of different molecules or accomplish concentration of like molecules.

The term “tangential flow filtration” refers to a specific filtration method in which a solute-containing solution passes tangentially across an ultrafiltration membrane and lower molecular weight solutes are passed through the membrane by applying pressure. The higher molecular weight solute-containing solution passing tangentially across the ultrafiltration membrane is retained, and thus this solution is referred to herein as “retentate.” The lower molecular weight solutes that pass through the ultrafiltration membrane are referred to herein as “permeate.” Thus, the retentate is concentrated by flowing along, e.g., tangentially, the surface of an ultrafiltration membrane under pressure. The ultrafiltration membrane has pore size with a certain cut off value. In some aspects, the cutoff value is about 50 kDa or less. In some aspects, the cutoff value is 30 kD or less.

The term “diafiltration” or “DF” refers to, for example, using an ultrafiltration membrane to remove, replace, or lower the concentration of solvents, buffers, and/or salts from solutions or mixtures containing proteins, peptides, nucleic acids, or other biomolecules.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. In some aspects, an antibody is a full-length antibody.

An immunoglobulin may derive from any of the commonly known isotypes, including but not limited to IgA, secretory IgA, IgG, IgD, IgE, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. The term “antibody” can include multivalent antibodies capable of binding more than two antigens (e.g., trivalent antibody). A trivalent antibody are IgG-shaped bispecific antibodies composed of two regular Fab arms fused via flexible linker peptides to one asymmetric third Fab-sized binding module. This third module replaces the IgG Fc region and is composed of the variable region of the heavy chain fused to CH3 with “knob”-mutations, and the variable region of the light chain fused to CH3 with matching “holes”. The hinge region does not contain disulfide bonds to facilitate antigen access to the third binding site. Where not expressly stated, and unless the context indicates otherwise, the term “antibody” includes monospecific, bispecific, or multi-specific antibodies, as well as a single chain antibody.

The term “antigen-binding portion” or “antigen-binding fragment” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An “isolated antibody” refers to an antibody that is substantially free of other antibodies having different antigenic specificities (e.g., an isolated antibody that binds specifically to PD-L1 is substantially free of antibodies that bind specifically to antigens other than PD-L1). An isolated antibody that binds specifically to PD-1 may, however, have cross-reactivity to other antigens, such as PD-L1 molecules from different species. Moreover, an isolated antibody can be substantially free of other cellular material and/or chemicals.

A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “culturing” refers to growing one or more cells in vitro under defined or controlled conditions. Examples of culturing conditions which can be defined include temperature, gas mixture, time, and medium formulation.

The term “inoculation” as used herein refers to the addition of cells to culture medium to start the culture.

The term “induction” or “induction phase” or “growth phase” of the cell culture as used herein refers to the initial seeding of the bioreactor (e.g., seed bioreactor) at the outset of upstream cell culture, and includes the period of exponential cell growth (for example, the log phase) where cells are primarily dividing rapidly. During this phase, the rate of increase in the density of viable cells is higher than at any other time point.

As used herein, the term “production phase” of the cell culture refers to the period of time during which cell growth is stationary or is maintained at a near constant level. The density of viable cells remains approximately constant over a given period of time. Logarithmic cell growth has terminated and protein production is the primary activity during the production phase. The medium at this time is generally supplemented to support continued protein production and to achieve the desired glycoprotein product.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

As used herein, the terms “culture medium” and “cell culture medium” and “feed medium” and “fermentation medium” refer to a nutrient solutions used for growing and or maintaining cells, especially mammalian cells. Without limitation, these solutions ordinarily provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The nutrient solution can be supplemented electively with one or more components from any of the following categories: (1) hormones and other growth factors such as, serum, insulin, transferrin, and epidermal growth factor; (2) salts, for example, magnesium, calcium, and phosphate; (3) buffers, such as HEPES; (4) nucleosides and bases such as, adenosine, thymidine, and hypoxanthine; (5) protein and tissue hydrolysates, for example peptone or peptone mixtures which can be obtained from purified gelatin, plant material, or animal byproducts; (6) antibiotics, such as gentamycin; (7) cell protective agents, for example pluronic polyol; and (8) galactose. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enz. 58:44 (1979), Barnes et al., Anal. Biochem. 102:255 (1980) can be used as culture media for the host cells. Any other necessary supplements can also be included at appropriate concentrations.

Various aspects of the disclosure are described in further detail in the following subsections.

### I. Counter-Current Dialysis

The present disclosure provides a highly effective approach to remove contaminants during protein purification using counter-current dialysis-in-series, without the need for chromatography. As such, the present disclosure provides methods for purifying a protein of interest that uses approximately 1/10^{th} the amount of water and solutions as chromatographic processes.

In some aspects, the present disclosure provides a method for purifying a protein of interest using counter-current concentration dialysis, comprising: (a) passing a first flow solution comprising the protein of interest and impurities into a first hollow fiber dialysis cassette at a first flow rate, wherein the dialysis cassette comprises a dialysate in-flow, at a dialysate in-flow rate, and a dialysate out-flow, at a dialysate out-flow rate; and wherein the first flow solution is counter-current to the dialysate in-flow and out-flow; (b) passing the impurities through a semi-permeable membrane of the dialysis cassette, wherein the dialysate in-flow rate is higher than the first flow rate, wherein a second flow solution comprising the protein of interest and a reduced level of impurities exits the dialysis cassette at a second flow rate, and wherein the dialysate out-flow rate is the sum of the dialysate in-flow rate and the difference between the first flow rate and the second flow rate; (c) optionally passing the second flow solution from the first dialysis cassette directly into a second dialysis cassette; and optionally repeating steps (a) and (b) with the second flow solution and the second dialysis cassette, thereby forming a third flow solution with a reduced level of impurities compared to the first and second flow solutions.

In some aspects, the method further comprises passing the third flow solution from the second dialysis cassette directly into a third dialysis cassette, and repeating steps (a) and (b), thereby forming a fourth flow solution with a reduced level of impurities compared to the first, second, and third flow solutions.

In some aspects, the method further comprises passing the fourth flow solution from the third dialysis cassette directly into a fourth dialysis cassette, and repeating steps (a) and (b), thereby forming a fifth flow solution with a reduced level of impurities compared to the first, second, third, and fourth flow solutions. In some aspects, the asymmetric dialysis at various manufacturing scales can be performed using hollow-fiber membranes with areas of 1.0 m², 2.0 m², 2.5 m², 3.6 m², 5.4 m², 7 m², 8 m², or 10 m².

In some aspects, the dialysate in-flow rate is about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 7, about 1.8, about 1.9, about 2.0, about 2.1, about 2.2, about 2.25, about 2.3, about 2.4, about 2.5, about 2.6, about 2.7, about 2.8, about 2.9, about 3, about 4, about 5, about 6, about 7, about 8, about 9, or about 10 times higher than the first flow rate. In some aspects, the dialysate in-flow rate is about 2.25 times higher than the first flow rate.

In some aspects, the second flow rate is about 0.1, about 0.15, about 0.2, about 0.25, about 0.3, about 0.35, about 0.4, about 0.45, about 0.5, about 0.55, about 0.6, about 0.65, about 0.7, or about 0.75 times the first flow rate. In some aspects, the second flow rate is between about 0.25 to about 0.5 times the first flow rate.

In some aspects, the first flow rate is between about 0.01 mL/minute to about 25 mL/minute. In some aspects, the first flow rate is about 0.5 mL/minute, about 1 mL/minute, about 2 mL/minute, about 3 mL/minute, about 4 mL/minute, about 5 mL/minute, about 6 mL/minute, about 7 mL/minute, about 8 mL/minute, about 9 mL/minute, about 10 mL/minute, about 11 mL/minute, about 12 mL/minute, about 13 mL/minute, about 14 mL/minute, about 15 mL/minute, about 16 mL/minute, about 17 mL/minute, about 18 mL/minute, about 19 mL/minute, about 20 mL/minute, about 21 mL/minute, about 22 mL/minute, about 23 mL/minute, about 24 mL/minute, or about 25 mL/minute. In some aspects, the second flow rate is about 1 mL/minute, about 2 mL/minute, about 3 mL/minute, about 4 mL/minute, about 5 mL/minute, about 6 mL/minute, about 7 mL/minute, about 8 mL/minute, about 9 mL/minute, about 10 mL/minute, about 11 mL/minute, about 12 mL/minute, about 13 mL/minute, about 14 mL/minute, about 15 mL/minute, about 16 mL/minute, about 17 mL/minute, about 18 mL/minute, about 19 mL/minute, about 20 mL/minute, about 21 mL/minute, about 22 mL/minute, about 23 mL/minute, about 24 mL/minute, about 25 mL/minute, about 26 mL/minute, about 27 mL/minute, about 28 mL/minute, about 29 mL/minute, about 30 mL/minute, about 31 mL/minute, about 32 mL/minute, about 33 mL/minute, about 34 mL/minute, about 35 mL/minute, about 36 mL/minute, about 37 mL/minute, about 38 mL/minute, about 39 mL/minute, about 40 mL/minute, about 41 mL/minute, about 42 mL/minute, about 43 mL/minute, about 44 mL/minute, about 45 mL/minute, about 46 mL/minute, about 47 mL/minute, about 48 mL/minute, about 49 mL/minute, or about 50 mL/minute.

In some aspects, the Asymmetric Continuous Counter-Current Concentration Dialysis reduces the level of impurities present in the solution comprising the protein of interest. In some aspects, removal of molecules with molecular weights 100 kDa, 200 kDa, 300 kDa, 500 kDa, 700 kDa, or 1000 kDa are removed from the mAb feed.

In some aspects, the impurities comprise host cell proteins (HCP). In some aspects, the Asymmetric Continuous Counter-Current Concentration Dialysis reduces the amount of HCP by about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In some aspects, the impurities comprise low molecular weight species. In some aspects, the methods described herein are capable of removing about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the low molecular weight species from the solution comprising the protein of interest.

In some aspects, the impurities comprise DNA. In some aspects, the Asymmetric Continuous Counter-Current Concentration Dialysis reduces the amount of DNA to about 20 pg/mL or lower, about 18 pg/mL or lower, about 16 pg/mL or lower, about 14 pg/mL or lower, about 12 pg/mL or lower, about 10 pg/mL or lower, about 8 pg/mL or lower, about 6 pg/mL or lower, about 4 pg/mL or lower, or about 2 pg/mL or lower.

In some aspects, the contaminant comprises residual Protein A. In some aspects, the Asymmetric Continuous Counter-Current Concentration Dialysis reduces residual Protein A by about about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In some aspects, the protein of interest is diafiltrated. In some aspects, the protein of interest is diafiltrated into a buffer comprising a pH of about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about, 6.9, about 7.0, about 7.1, about 7.2, about 7.3, about 7.4, about 7.5, about 7.6, about 7.7, about 7.8, about 7.9, or about 8. In some aspects, the protein of interest is diafiltrated into a buffer comprising a pH of about 6. In some aspects, the protein of interest is diafiltrated into a buffer comprising a conductivity of about 1.0 mS/cm. In some aspects, the protein of interest is diafiltrated into a buffer comprising a conductivity of least 0.5 mS/cm, at least 1.0 mS/cm, at least 1.5 mS/cm, at least 2.0 mS/cm, at least 2.5 mS/cm, at least 3.0 mS/cm, at least 3.5 mS/cm, at least 4.0 mS/cm, at least 4.5 mS/cm, or at least 5.0 mS/cm. In some aspects, the protein of interest is diafiltrated into a buffer comprising a pH of about 6 and conductivity of about 1.0 mS/cm. In some aspects, the method is used for exchanging other carbohydrates such as sucrose, trehalose, lactose, maltose etc. or sugar alcohols such as mannitol, sorbitol, xylitol, lactitol, maltitol etc. into the product containing the protein of interest.

In some aspects, the protein of interest is obtained from a bioreactor. In some aspects, the protein of interest is obtained from a bioreactor at a concentration of about 10 g/L to about 100 g/L. In some aspects, the protein of interest is obtained from a bioreactor at a concentration of about 15 g/L to about 95 g/L. In some aspects, the protein of interest is obtained from a bioreactor at a concentration of about 20 g/L to about 40 g/L. In some aspects, the protein of interest is obtained from a bioreactor at a concentration of about 25 g/L to about 35 g/L. In some aspects, the protein of interest is obtained from a bioreactor at a concentration of about 30 g/L to about 35 g/L. In some aspects, the protein of interest is obtained from a bioreactor at a concentration of about 25 g/L to about 30 g/L.

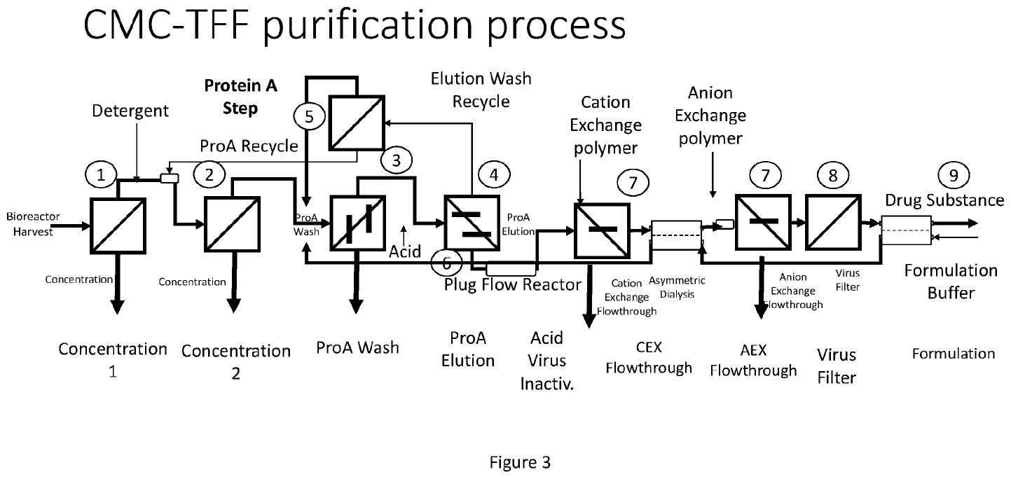

In some aspects, the protein of interest is obtained following an ultrafiltration step. In some aspects, the ultrafiltration step is combined with the dialysis step and performed simultaneously (e.g., Asymmetric Continuous Counter-Current Concentration Dialysis-in-series, FIG. 3).

In some aspects, the methods described herein can purify about 0.1 kg/day, about 0.5 kg/day, about 1 kg/day, about 2 kg/day, about 3 kg/day, about 4 kg/day, about 5 kg/day, about 6 kg/day, about 7 kg/day, about 8 kg/day, about 9 kg/day or about 10 kg/day of protein of interest.

### III. Protein of Interest

In some aspects, the methods disclosed herein can be applied to any protein product (e.g., a protein of interest). In some aspects, the protein product is a therapeutic protein. In some aspects, the therapeutic protein is selected from an antibody or antigen-binding fragment thereof, an Fc fusion protein, an anticoagulant, a blood clotting factor, an engineered protein scaffold, an enzyme, a growth factor, a hormone, an interferon, an interleukin, a receptor, and a thrombolytic. In some aspects, the protein product is an antibody or antigen-binding fragment thereof. In some aspects, the protein is a recombinant protein.

In other embodiments, the protein of interest is produced in a host cell. In some embodiments, the protein of interest is produced in culture comprising mammalian cells. In some embodiments, the mammalian cells are Chinese hamster ovary (CHO) cells, HEK293 cells, mouse myeloma (NS0), baby hamster kidney cells (BHK), monkey kidney fibroblast cells (COS-7), Madin-Darby bovine kidney cells (MDBK) or any combination thereof. In some embodiments, the starting mixture can be a harvested cell culture fluid, a cell culture supernatant, a conditioned cell culture supernatant, a cell lysate, and a clarified bulk.

In some aspects, the protein product is an antibody or an antigen binding fragment thereof. In some aspects, the protein product is a chimeric polypeptide comprising an antigen binding fragment of an antibody. In certain embodiments, the protein product is a monoclonal antibody or an antigen binding fragment thereof (“mAb”). The antibody can be a human antibody, a humanized antibody, or a chimeric antibody. In certain embodiments, the protein product is a bispecific antibody.

In some aspects, a mixture comprising the protein product and the contaminant comprises a product of a prior purification step. In some aspects, the mixture is the raw product of a prior purification step. In some aspects, the mixture is a solution comprising the raw product of a prior purification step and a buffer, e.g., the starting buffer. In some aspects, the mixture comprises the raw product of a prior purification step reconstituted in the starting buffer.

In some aspects, the source of the protein product is bulk protein. In some aspects, the source of the protein product is a composition comprising protein product and non-protein components. The non-protein components can include DNA and other contaminants.

In some aspects, the source of the protein product is from an animal. In some aspects, the animal is a mammal such as a non-primate (e.g., cow, pig, horse, cat, dog, rat etc.) or a primate (e.g., monkey or human). In some aspects, the source is tissue or cells from a human. In certain aspects, such terms refer to a non-human animal (e.g., a non-human animal such as a pig, horse, cow, cat or dog). In some aspects, such terms refer to a pet or farm animal. In some aspects, such terms refer to a human.

In some aspects, the protein products purified by the methods described herein are fusion proteins. A “fusion” or “fusion protein” comprises a first amino acid sequence linked in frame to a second amino acid sequence with which it is not naturally linked in nature. The amino acid sequences which normally exist in separate proteins can be brought together in a fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some aspects, the proteins purified by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In some aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some aspects, antibodies described herein are IgG antibodies, or a class (e.g., human IgG1 or IgG4) or subclass thereof. In a aspects, the antibody is a humanized monoclonal antibody. In some aspects, the antibody is a human monoclonal antibody, preferably that is an immunoglobulin. In some aspects, an antibody described herein is an IgG1, or IgG4 antibody.

The present disclosure is directed to methods disclosed herein, wherein the protein of interest is an antibody, an antigen-binding antibody fragment, a fusion protein, a naturally occurring protein, a chimeric protein, or any combination thereof. In some aspects, the protein of interest is a full length IgG antibody. In some aspects, the antibody is an IgG1, IgG2, IgG3, and/or IgG4, or hybrids thereof. In some aspects, the antibody is a therapeutic antibody.

In some aspects, the methods disclosed herein are accomplished using bacterial cells, yeast cells, insect cells, or mammalian cells. In some aspects, the mammalian cells are Chinese hamster ovary cells. In some aspects, the protein of interest are prepared by the methods disclosed herein.

The present disclosure is further illustrated by the following examples which should not be construed as further limiting.

### EXAMPLES

### Example 1—Systems and Methods for Unified Concentration and Buffer Exchange

### Proteins and Membranes

A feed containing a monoclonal antibody (mAb) was adjusted to pH 5.0, 50 mM Sodium acetate buffer, and salinity of 200 mM NaCl (conductivity, 20+3 mS/cm) to mimic the cation exchange elution pool. The mAb concentration in the feed was adjusted between 7-30 g/L. The hollow fiber modules used in this study were composed of polysulfone or polyethersulfone. The polysulfone membranes used are Optiflux 180NR (Fresenius, USA) and Renaflow HF1200 (Minntech, USA), with a membrane surface area of 1.8 m² and 1.2 m², respectively. The polyethersulfone membrane, Minikros 0.16 m² (S04-E030-05-N) was purchased from Repligen, USA. Each study used a new hollow fiber module except the Minikros module, which was cleaned with 0.1 N sodium hydroxide for 20 mins before reusing.

### Asymmetric Dialysis Method

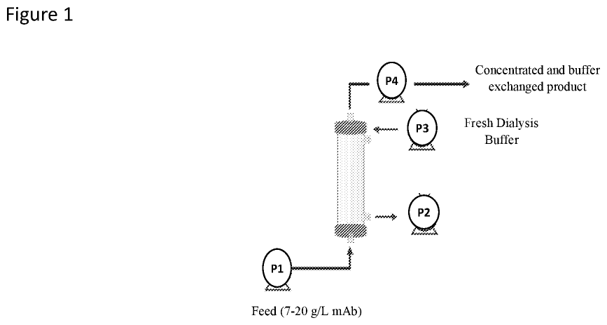

The hollow fiber module was mounted in a vertical orientation where the feed was introduced from the bottom port on the lumen-side through pump P1 (FIG. 1). The shell-side port proximal to the feed port (shell-outlet) was attached to pump P2. The distal shell-side (shell-inlet) and lumen-side (lumen-outlet) ports were attached to pumps P3 and P4, respectively. The dialysis buffer was introduced into the shell side using pump P3 while the pump P2 modulated the flow rate at the shell outlet. The concentrated and buffer exchanged product was collected at pump P4. The peristaltic pumps, P1, P2, P3, and P4, equipped with appropriate pump heads and tubing, were calibrated by timed collection using a digital balance before starting the process.

Pressures were monitored using Pendotech pressure sensors placed immediately before and after the inlet/outlet ports. The shell and lumen compartments of hollow-fiber module (1.8 m², OptiFlux 180NR) were flushed with dialysis buffer, 20 mM Histidine pH 5.7±1 before the experiment. All the solutions used in the experiment were filtered through a 0.45 um PES filter. In a typical experimental setup with feed flow (P1) of 20 ml/min (Flux 0.7 LMH), the pump P4 was adjusted to 5 mL/min to attain a 4×concentration factor along the hollow fiber membrane. While on the shell-side, the pumps P2 and P3 were adjusted to 60 mL/min and 45 mL/min, respectively. Therefore, to allow simultaneous product concentration and buffer exchange, the flow rates on all the pumps were modulated. The companion inlet and outlet flow rates for feed and the exchange buffer were proportionally paired but not matched (Table 1). All experiments were performed at room temperature (22±2° C.) without deliberate temperature control. For some experiments, vitamin B12 was dissolved in the preconditioned mAb feed as a model impurity. All experiments were performed in single-pass mode with no feed or dialysis buffer recirculation. Small samples were collected periodically from the lumen outlet for off-line pH, conductivity, mAb, and histidine concentration.

TABLE 1
Flow rate parameters for a typical asymmetric dialysis setup

| Feed | Shell- | Shell- | Lumen- | Estimated |
|---|---|---|---|---|
| Flow | outlet | inlet | outlet | concentration |
| (P1, | (P2, | (P3, | (P4, | factor |
| mL/min) | mL/min) | mL/min) | mL/min) | (P1/P2) |
| 20 | 60 | 45 | 5 | 4x |
| 25 | 76.25 | 56.25 | 5 | 5x |
| 45 | 141.75 | 101.25 | 4.5 | 10x |

In certain experiments, the pump P4 was not used or replaced with a backpressure regulator to attain the desired concentration factor.

### Strategy to Reduce Buffer Consumption

The buffer utilization in asymmetric dialysis is dependent on the operating α′, where,

$${α}^{′} = \frac{Dialysis buffer flow rate}{Product flow rate}$$

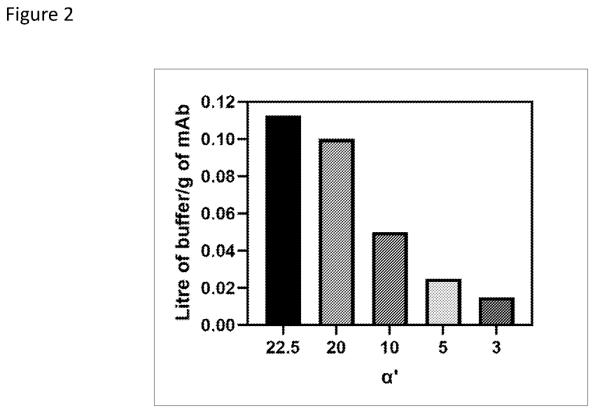

Several values of α′ were evaluated to reduce the buffer consumption. At α′=5, there was a 75% reduction in buffer consumption without impacting the process performance. Whereas the asymmetric dialysis process with an α′ value of 22.5 and feed mAb at 20 g/L and a concentration factor of 10× the resultant buffer consumption will be >0.1 L/g, mAb (FIG. 2).

### Results

### Example 2

In this example, a 30 g/L mAb feed (pH 5, 200 mM NaCl) was supplied to the hollow fiber using pump P1 at the flow rate of 20 ml/min (0.7 LMH), the pump P4 was adjusted to 5 mL/min for a desired 4×concentration factor along the hollow fiber membrane. For the shell-side, the dialysis buffer pumps P2 and P3 were adjusted to 60 mL/min and 45 mL/min, respectively. As shown in Table 2, both Fresenius and Repligen hollow-fibers showed comparable salt removal and buffer exchange performance.

TABLE 2
Asymmetric dialysis performance at 30 g/L mAb feed

| | Fresenius | Repligen |
|---|---|---|
| mAb product attributes | | |
| pH | 6.1 | 6.0 |
| Conductivity (mS/cm) | 1.4 | 1.6 |
| Histidine Concentration (mM) | 15.0 | 15.4 |
| Process Attributes | | |
| Concentration Factor | 3.7x | 4.0x |
| TMP (psi) | 0.2 | 0 |

### Example 3

In this example, a 20 g/L mAb feed (pH 5, 200 mM NaCl) was supplied to the hollow fiber using pump P1 at the flow rate of 45 ml/min (1.5 LMH), the pump P4 was adjusted to 4.5 mL/min for a desired 10×concentration factor along the hollow fiber membrane. For the shell-side, the dialysis buffer pumps P2 and P3 were adjusted to 141.75 mL/min and 101.25 mL/min (20 mM Histidine pH 5.9), respectively.

### Example 4

In this example, in order to reduce buffer consumption a lower α′ of 5 was selected where, a 20 g/L mAb feed (pH 5, 200 mM NaCl) was supplied to the hollow fiber device using pump P1 at the flow rate of 45 ml/min (1.5 LMH), the pump P4 was adjusted to 4.5 mL/min for a desired 10×concentration factor along the hollow fiber membrane. For the shell-side, the dialysis buffer pumps P2 and P3 were adjusted to 63 mL/min and 22.5 mL/min, respectively.

### Example 5

In this example, a relatively large model impurity/tracer was removed from the feed using asymmetric dialysis with the α′ of 5. For this evaluation, a 20 g/L mAb feed (pH 4.9, 200 mM NaCl) with 3.2 g/L of Vitamin B12 (˜1,356 kDa) was supplied to the hollow fiber device using pump P1 at the flow rate of 45 ml/min (1.5 LMH), the pump P4 was adjusted to 4.5 mL/min for a desired concentration factor along the hollow fiber membrane. For the shell-side, the dialysis buffer pumps P2 and P3 were adjusted to 63 mL/min and 22.5 mL/min, respectively. From empirical experimental data pertaining to this evaluation, the fresh dialysis buffer flow rate was adjusted to pH 5.6 to achieve target product pH of 6.0.

TABLE 3
Asymmetric dialysis performance at 20 g/L
mAb feed with model impurity vitamin B12

| | mAb | | Conductivity |
|---|---|---|---|
| | (g/L) | pH | (mS/cm) |
| Feed | 20 | 4.9 | 22.2 |
| Dialysis buffer (20 mM Histidine) | — | 5.6 | 1.4 |
| Product pool | 183 | 6.0 | 1.9 |
| Vitamin B12 Removal | 99.7% | | |

### Example 6

In this example, a non-ionic molecule such as carbohydrate, glucose was exchanged into the product using asymmetric dialysis with the α′ of 5 and dialysis buffer containing glucose (4.6 g/L). For this evaluation, a 20 g/L mAb feed (pH 4.9, 200 mM NaCl) was supplied to the hollow fiber device using pump P1 at the flow rate of 45 ml/min (1.5 LMH), the pump P4 was adjusted to 4.5 mL/min for a desired concentration factor along the hollow fiber membrane. For the shell-side, the dialysis buffer pumps P2 and P3 were adjusted to 63 mL/min and 22.5 mL/min, respectively.

TABLE 4
Asymmetric dialysis performance at 20 g/L
mAb feed with glucose in dialysis buffer

| | mAb | | Conductivity | Glucose |
|---|---|---|---|---|
| | (g/L) | pH | (mS/cm) | (g/L) |
| Feed | 19 | 5.0 | 23.2 | — |
| Dialysis buffer (20 mM Histidine) | — | 5.6 | 1.6 | 4.6 |
| Product pool | 175 | 6.0 | 1.9 | 4.4 |

### Example 7

In this example, an ionic compound such as NaCl, was exchanged into the product using asymmetric dialysis with the α′ of 5 and dialysis buffer, 20 mM Histidine pH 5.9, 100 mM NaCl. For this evaluation, a low conductivity (1.2 mS/cm) 20 g/L mAb feed (20 mM histidine pH 6.0) was supplied to the hollow fiber device using pump P1 at the flow rate of 45 ml/min (1.5 LMH), the pump P4 was adjusted to 4.5 mL/min for a desired concentration factor along the hollow fiber membrane. For the shell-side, the dialysis buffer pumps P2 and P3 were adjusted to 63 mL/min and 22.5 mL/min, respectively.

| | mAb | | Conductivity |
|---|---|---|---|
| | (g/L) | pH | (mS/cm) |
| Feed | 20 | 6.0 | 1.2 |
| Dialysis buffer (20 mM Histidine, 0.1M NaCl) | — | 5.9 | 11.7 |
| Product pool | 175 | 6.0 | 1.9 |

Thus, the above data demonstrates the development of an innovative one-step process, asymmetric dialysis, for continuous UF and buffer exchange eliminating the need for two-step UF/DF. Careful manipulation of inlet/outlet flow rates are performed to achieve product concentration, buffer exchange, and salt removal. Product concentrations 105 g/L (3.8×), 200 g/L (10×), 64 g/L (9.4×) starting from feed concentrations of 28 g/L, 20 g/L, and 7 g/L, respectively, can be achieved as described above with modest (<6 psi) pressure profile across the cartridge. The method also achieves reduced buffer utilization by 74% (0.026 L/g, mAb) compared to conventional batch UF-DF (0.1 L/g, mAb) with mAb productivities up to 0.7 kg/m²/day. This provides a simplified and smaller footprint compared to current generation technologies.

What is claimed is:

1. A method for purifying a protein of interest using counter-current concentration dialysis, comprising:
(a) passing a first flow solution comprising the protein of interest and impurities into a first hollow fiber dialysis cassette at a first flow rate, wherein the dialysis cassette comprises a dialysate in-flow, at a dialysate in-flow rate, and a dialysate out-flow, at a dialysate out-flow rate; and wherein the first flow solution is counter-current to the dialysate in-flow and out-flow;
(b) passing the impurities through a semi-permeable membrane of the dialysis cassette, wherein the dialysate in-flow rate is higher than the first flow rate, wherein a second flow solution comprising the protein of interest and a reduced level of impurities exits the dialysis cassette at a second flow rate, and wherein the dialysate out-flow rate is the sum of the dialysate in-flow rate and the difference between the first flow rate and the second flow rate;
(c) optionally passing the second flow solution from the first dialysis cassette directly into a second dialysis cassette; and
(d) optionally repeating steps (a) and (b) with the second flow solution and the second dialysis cassette, thereby forming a third flow solution with a reduced level of impurities compared to the first and second flow solutions.

2. The method of claim 1, further comprising passing the third flow solution from the second dialysis cassette directly into a third dialysis cassette, and repeating steps (a) and (b), thereby forming a fourth flow solution with a reduced level of impurities compared to the first, second, and third flow solutions.

3. The method of claim 2, further comprising passing the fourth flow solution from the third dialysis cassette directly into a fourth dialysis cassette, and repeating steps (a) and (b), thereby forming a fifth flow solution with a reduced level of impurities compared to the first, second, third, and fourth flow solutions.

4. The method of any one of claims 1-3, wherein the dialysate in-flow rate is about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 7, about 1.8, about 1.9, about 2.0, about 2.1, about 2.2, about 2.25, about 2.3, about 2.4, about 2.5, about 2.6, about 2.7, about 2.8, about 2.9, about 3, about 4, about 5, about 6, about 7, about 8, about 9, or about 10 times higher than the first flow rate.

5. The method of claim 4, wherein the dialysate in-flow rate is about 2.25 times higher than the first flow rate.

6. The method of any of claims 1-5, wherein the second flow rate is about 0.1, about 0.15, about 0.2, about 0.25, about 0.3, about 0.35, about 0.4, about 0.45, about 0.5, about 0.55, about 0.6, about 0.65, about 0.7, or about 0.75 times the first flow rate.

7. The method of any one of claims 1-6, wherein the second flow rate is between about 0.25 to about 0.5 times the first flow rate.

8. The method of any one of claims 1-7, wherein the first flow rate is between about 0.01 mL/minute to about 25 mL/minute.

9. The method of claim 8, wherein the first flow rate is about 0.5 mL/minute, about 1 mL/minute, about 2 mL/minute, about 3 mL/minute, about 4 mL/minute, about 5 mL/minute, about 6 mL/minute, about 7 mL/minute, about 8 mL/minute, about 9 mL/minute, about 10 mL/minute, about 11 mL/minute, about 12 mL/minute, about 13 mL/minute, about 14 mL/minute, about 15 mL/minute, about 16 mL/minute, about 17 mL/minute, about 18 mL/minute, about 19 mL/minute, about 20 mL/minute, about 21 mL/minute, about 22 mL/minute, about 23 mL/minute, about 24 mL/minute, or about 25 mL/minute.

10. The method of any one of claims 1-9, wherein the impurities comprise low molecule weight species.

11. The method of any one of claims 1-10, wherein the protein of interest is diafiltrated.

12. The method of any one of claims 1-11, wherein the protein of interest is obtained from a bioreactor.

13. The method of any one of claims 1-12, wherein about 0.1 kg/day, about 0.5 kg/day, about 1 kg/day, about 2 kg/day, about 3 kg/day, about 4 kg/day, about 5 kg/day, about 6 kg/day, about 7 kg/day, about 8 kg/day, about 9 kg/day or about 10 kg/day of protein of interest is purified.

14. The method of any one of claims 1-13, wherein the protein of interest comprises an antibody, an antigen binding fragment, a fusion protein, a naturally occurring protein, a chimeric protein, or any combination thereof.

15. The method of claim 14, wherein the protein comprises an antibody selected from IgM, IgA, IgE, IgD, and IgG.

16. The method of claim 15, wherein the protein comprises an antibody and the antibody is an IgG antibody selected from IgG1, IgG2, IgG3, and IgG4.

17. The method of claim 16, wherein the antibody is a therapeutic antibody.
